(19) United States
(12) Patent Application Publication
(10) Pub. No.: US 2025/0280900 A1
(43) Pub. Date: Sep. 11, 2025

(54) FOLDABLE GARMENTS

(71) Applicant: Goest Hagues Pty Ltd, Mount Warrigal (AU)

(72) Inventors: Nina Brigitte Smith, Houston, TX (US); Floriane Lena Goest, Mount Warrigal, NSW (AU)

(21) Appl. No.: 19/214,649

(22) Filed: May 21, 2025

Related U.S. Application Data
(63) Continuation of application No. PCT/US2023/081295, filed on Nov. 28, 2023.
(60) Provisional application No. 63/385,941, filed on Dec. 2, 2022.

(51) Int. Cl. A41B 9/00; A61F 13/496

(52) U.S. Cl. CPC A41B 9/004; A41B 9/001; A61F 13/496; A41B 2300/52; A41B 2400/62

(57) ABSTRACT

A garment that can be folded in on itself to form a self-contained pouch and unfolded to form a wearable garment is provided. In one aspect, the garment is an undergarment that includes a front panel, a first layer, a second layer, and a circular seam. The first layer can be coupled to the front panel along edges of the right portion of the front panel. The second layer can be coupled to the front panel along edges of the left portion of the front panel. The circular seam can join the first layer, the second layer, and the front panel. The circular seam can be located in the central portion of the front panel where the first layer overlaps with the second layer. An edge of the first layer can form an opening within the circular seam, through which the undergarment can be folded in on itself. The circular seam can define a shape of the self-contained pouch when the undergarment is folded in on itself.

### CROSS REFERENCE TO RELATED APPLICATIONS

This application is a continuation of International Application No. PCT/US2023/081295, filed Nov. 28, 2023, which claims the benefit of U.S. Provisional Application No. 63/385,941, filed Dec. 2, 2022, the content of each of which is hereby incorporated by reference in its entirety.

### BACKGROUND

### Field

The devices and methods disclosed herein are directed to an absorbent undergarment that is designed to fold in on itself to form a self-contained pouch. The disclosure is not limited to undergarments and can be implemented in many types of garments, including but not limited to period or menstrual fluid absorbing garments, reusable diapers, garments for individuals with incontinence, swimwear, swim briefs, briefs, boxer briefs, women's garments, men's garments, unisex garments, and athletic attire (such as but not limited to cycling shorts and yoga attire).

### Description of the Related Art

Current absorbent undergarments do not provide for adequate post-wear management. When users are away from the home and have a need to store the undergarments prior to washing them, for example when traveling, there is no efficient and/or sanitary way to store the used and/or soiled undergarment. Period underwear may be capable of holding and/or absorbing a quantity of fluid, but the quantity of fluid is not contained or stored in a controlled manner. For example, this can rub and/or spill and/or leak onto other articles if not managed carefully.

### SUMMARY OF CERTAIN ILLUSTRATIVE EMBODIMENTS

In various embodiments, a garment according to the present disclosure can be folded in on itself to form a self-contained pouch and un-folded to form a wearable garment. The garment can be an undergarment. The undergarment can include a front panel, a first layer, a second layer, and a circular seam. The front panel can include an interior side and an exterior side. The exterior side can include a right portion and a left portion. The first layer can be coupled to the front panel along edges of the right portion of the front panel. The second layer can be coupled to the front panel along edges of the left portion of the front panel. The first layer can overlap with the second layer in a central portion of the exterior side of the front panel. The circular seam can join the first layer, the second layer, and the front panel. The circular seam can be located in the central portion of the front panel where the first layer overlaps with the second layer. The circular seam can define a shape of the self-contained pouch when the undergarment is folded in on itself.

In some embodiments, a portion of a leftmost edge of the first layer forms an opening within the circular seam.

In some embodiments, the portion of the leftmost edge of the first layer forming the opening is curved. In some embodiments, the edge of the first layer forming the opening can include a curve, such as a slight curve. In some embodiments, the edge of the first layer forming the opening is non-linear.

In some embodiments, a portion of the leftmost edge of the first layer forms an opening within the circular seam, the leftmost edge of the first layer is offset a distance from the center of the circular seam, and the opening is offset the distance from the center of the circular seam.

In some embodiments, the circular seam is centered relative to a right and a left side of the undergarment.

In some embodiments, the circular seam passes through a portion of the front panel.

In some embodiments, the circular seam does not pass through the interior side of the front panel.

In some embodiments, the front panel includes at least an exterior front panel layer and an interior front panel layer, wherein the circular seam passes through the exterior front panel layer but not the interior front panel layer.

In some embodiments, the undergarment is a re-usable and washable undergarment configured to absorb menstrual and other bodily fluids.

In another example embodiment, a garment can include a main body structure, two layers of fabric, and a pocket guide. The main body structure can include a front portion and a back portion. The two layers of fabric can be coupled to the front portion along outer edges of the front portion. The pocket guide can be configured to form a self-contained pouch when the garment is reversibly folded in on itself.

In some embodiments, the two layers of fabric overlap in a central region of the front portion, or the two layers of fabric do not overlap in a central region of the front portion.

In some embodiments, the two layers of fabric are coupled to an exterior side of the front portion or the two layers of fabric are coupled to an interior side of the front portion.

In some embodiments, the pocket guide is circular, elliptical, rectangular, square-shaped, heart-shaped, teardrop-shaped, or oval-shaped.

In some embodiments, the pocket guide is circular.

In some embodiments, an opening of the pocket guide forms a chord that does not pass through a center of the pocket guide. In some embodiments, an opening of the pocket guide forms a diameter of the pocket guide.

In some embodiments, a portion of an edge of one of the two layers forms an opening of the pocket guide, and the portion of the edge is curved. In some embodiments, the edge of the opening of the pocket guide can include a curve, such as a slight curve. In some embodiments, the edge of the opening of the pocket guide is non-linear.

In some embodiments, the pocket guide is centered between a right outer edge and a left outer edge of the front portion or the pocket guide is not centered between a right outer edge and a left outer edge of the front portion.

In some embodiments, the pocket guide is a seam or weld that joins the two layers of fabric and the front portion of the main body structure.

In some embodiments, the seam or weld passes through the front portion to an interior-most side of the front portion or the seam or weld does not pass through the front portion to an interior-most side of the front portion.

In some embodiments, the main body structure is a single unitary structure or the main body structure includes a plurality of portions joined together.

In some embodiments, the garment includes a washable material configured to absorb body fluids.

### BRIEF DESCRIPTION OF THE DRAWINGS

The disclosed aspects will hereinafter be described in conjunction with the accompanying drawings, provided to illustrate and not to limit the disclosed aspects, wherein like designations denote like elements.

FIG. 1A illustrates a front view of an example undergarment according to the present disclosure.

FIG. 1B illustrates a front view of the undergarment of FIG. 1A with layers removed to illustrate a front of the undergarment.

FIG. 2A illustrates a front view of another example undergarment according to the present disclosure.

FIG. 2B illustrates a front view of the undergarment of FIG. 2A being worn by a user.

FIG. 2C illustrates a rear perspective view of the undergarment of FIG. 2A.

FIGS. 2D and 2E illustrate side views of the undergarment of FIG. 2A.

FIG. 2F illustrates the undergarment of FIG. 2A folded in on itself to form a self-contained pouch.

FIG. 3 illustrates an example method of folding the undergarment of FIG. 2A-2E according to the present disclosure.

FIG. 4 illustrates a front view of another example undergarment according to the present disclosure.

### DETAILED DESCRIPTION

While the present description sets forth specific details of various embodiments, it will be appreciated that the description is illustrative only and should not be construed in any way as limiting. Furthermore, various applications of such embodiments and modifications thereto, which may occur to those who are skilled in the art, are also encompassed by the general concepts described herein. Each and every feature described herein, and each and every combination of two or more of such features, is included within the scope of the present disclosure provided that the features included in such a combination are not mutually inconsistent.

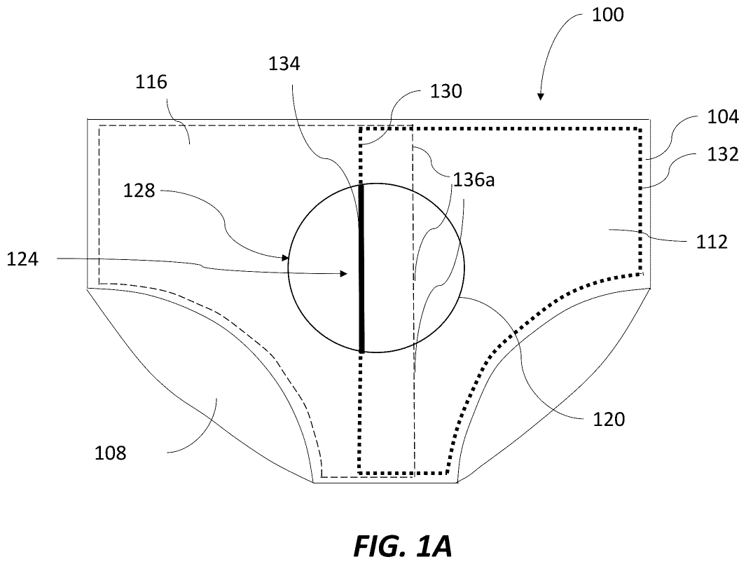

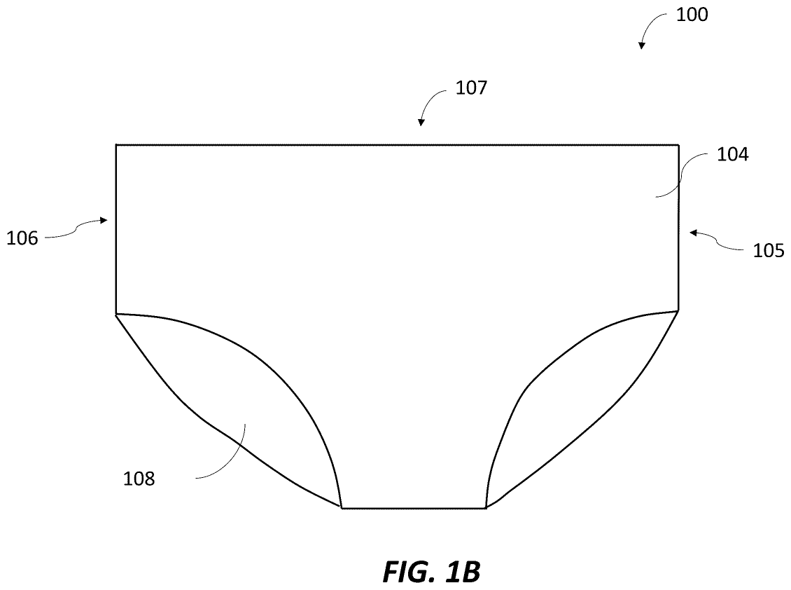

An example garment will now be described with reference to FIGS. 1A and 1B. The garment can be an undergarment. The disclosure is not limited to the undergarments depicted and described and can be implemented in many types of garments, including but not limited to, period or menstrual fluid absorbing garments, reusable diapers, garments for individuals with incontinence, swimwear, swim briefs, briefs, boxer briefs, women's garments, men's garments, and unisex garments, and athletic attire (such as but not limited to cycling shorts and yoga attire). The garments can include women's garments, men's garments, and unisex garments. FIG. 1A illustrates a front view of a garment 100 according to the present disclosure. In this non-limiting example, the garment is an undergarment 100. FIG. 1B illustrates a front view of the undergarment 100 with certain layers omitted in order to illustrate a front panel of the undergarment 100. Advantageously, the undergarment 100 includes a reversible pocket into which the entire under garment can be folded in on itself to obtain a self-contained pouch, and then unfolded back into the shape of a wearable undergarment.

Garments according to the present disclosure can absorb many kinds of bodily fluids, including but not limited to menstrual fluid, bodily discharges (for example, cervical discharge, mucus, and urine), and sweat. Example garments are described herein with reference to garments configured to absorb and/or contain menstrual fluid (sometimes referred to as period underwear), but the present disclosure is not limited to garments configured to absorb and/or contain menstrual fluid.

Garments according to the present disclosure can be configured to absorb relatively large quantities of bodily fluids. Embodiments of garments according to the present disclosure can be formed of materials that advantageously absorb fluids at a higher rate and a higher volume than standard materials. In one non-limiting embodiment, a garment according to the present disclosure is configured to absorb ten times its weight in under two seconds. In another non-limiting embodiment, a garment according to the present disclosure is configured to absorb fluids twenty times faster than garments formed of standard materials (such as but not limited materials including hemp, cotton, and hemp knits). In some embodiments of the present disclosure, some or all portions of the garment are formed of a super absorbent material, such as a super absorbent fabric. In one embodiment, the gusset of the garment is formed of a super absorbent fabric, while other portions are formed of a second, different material that is less absorbent that than the super absorbent fabric. In another embodiment, each of the portions of the garment are formed of a super absorbent material, such as a super absorbent fabric. A non-limiting example of a super absorbent fabric implemented in a garment of the present disclosure is Zorb® fabric by Wazoodle Fabrics, Bensalem, PA.

It will be understood that materials implemented in garments according to the present disclosure can be selected to achieve a desired absorbency volume and/or rate. For example, one garment configured for light absorbency scenarios can include a gusset formed of or lined with a material of a first thickness and/or a first absorbency rate, while another garment configured for heavy absorbency scenarios can include a gusset formed of or lined with a material of a second thickness greater than the first thickness and/or a second absorbency rate greater than the first absorbency rate.

Advantageously, garments according to the present disclosure can retain the absorbed fluids within the fabric of the garment while the garment is an unfolded configuration and/or a folded configuration. In one embodiment, fluids absorbed within the fabric of the garment do not leak out of the fabric while a user is wearing the garment, as the user removes or takes off the garment, during a process in which the user folds the garment from an unfolded configuration to a folded configuration, during a time that the garment remains in the folded configuration, and/or during a process in which the user unfolds the garment from the folded configuration to an unfolded configuration. In one example, absorbed fluids are not released from the garment while the user handles, folds, and unfolds the garment, and are released from the garment when the user washes the garment.

The undergarment 100 can have any general overall shape. For example, the undergarment 100 can take on the shape of any type of underwear, with non-limiting examples including hipster cut, full brief, midi cut, thong, and boxer briefs. The undergarment 100 can include a front panel 104 and a back panel 108. In some embodiments, the main body of the undergarment 100 can be a single unitary structure. In some embodiments, the main body structure can include a plurality of portions joined together, for example, the front panel or front portion 104 can be joined to the back panel or back portion 108. Portions of the undergarment 100 can be joined using any suitable method, including but not limited to sewing, gluing, or welding the portions together.

FIG. 1A illustrates a front view of the undergarment 100 including a first layer 112 and a second layer 116. FIG. 1B illustrates a front view of the undergarment 100 where the first layer 112 and second layer 116 have been omitted in order to illustrate portions of the front panel 104 that are not ordinarily visible after the first and second layers 112, 116 are joined to the front panel 104. The front panel includes a right portion 105, a left portion 106, and a central portion 107, as shown in FIG. 1B.

The undergarment 100 can include a first layer 112 and a second layer 116. The first layer 112 can be coupled or joined to the front panel 104 along edges of the right portion 105 of the front panel 104. The second layer 116 can be coupled or joined to the front panel 104 along edges of the left portion 106 of the front panel 104. In some embodiments, the edges of the pocket layers 112, 116 can align with the edges of the front panel 104 such that the seams are not visible when worn under the user's clothing.

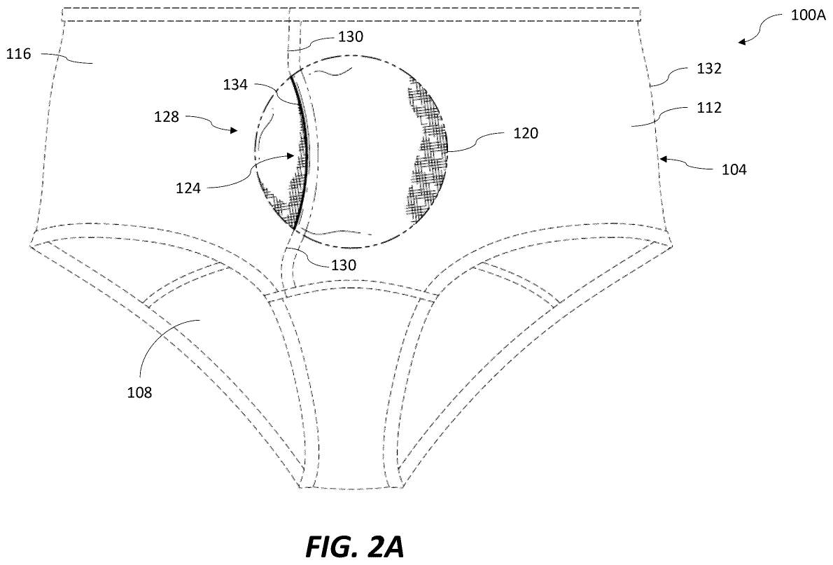

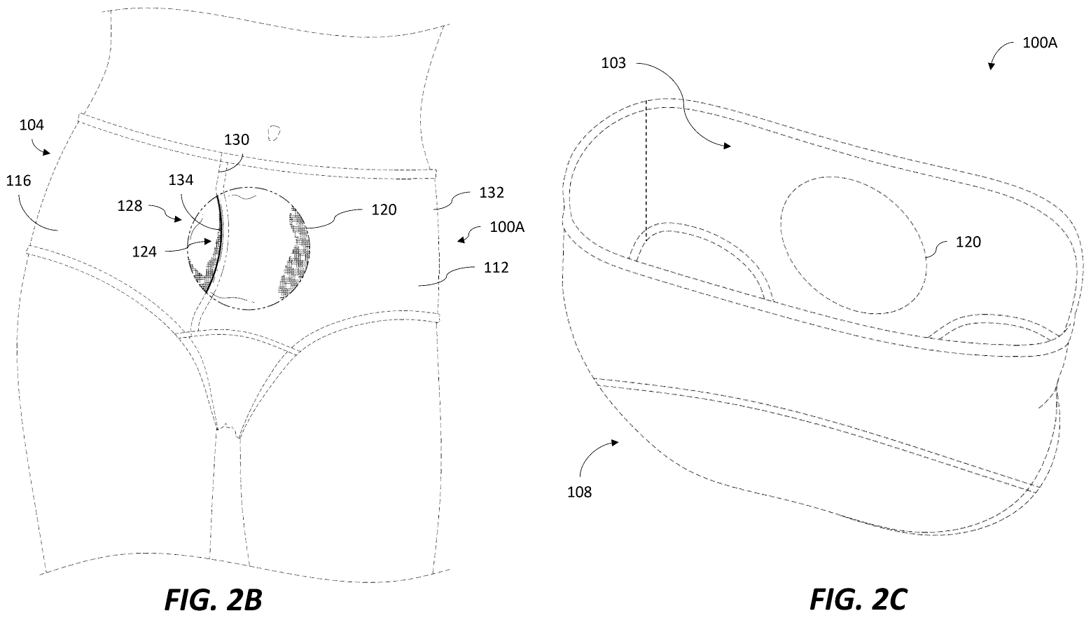

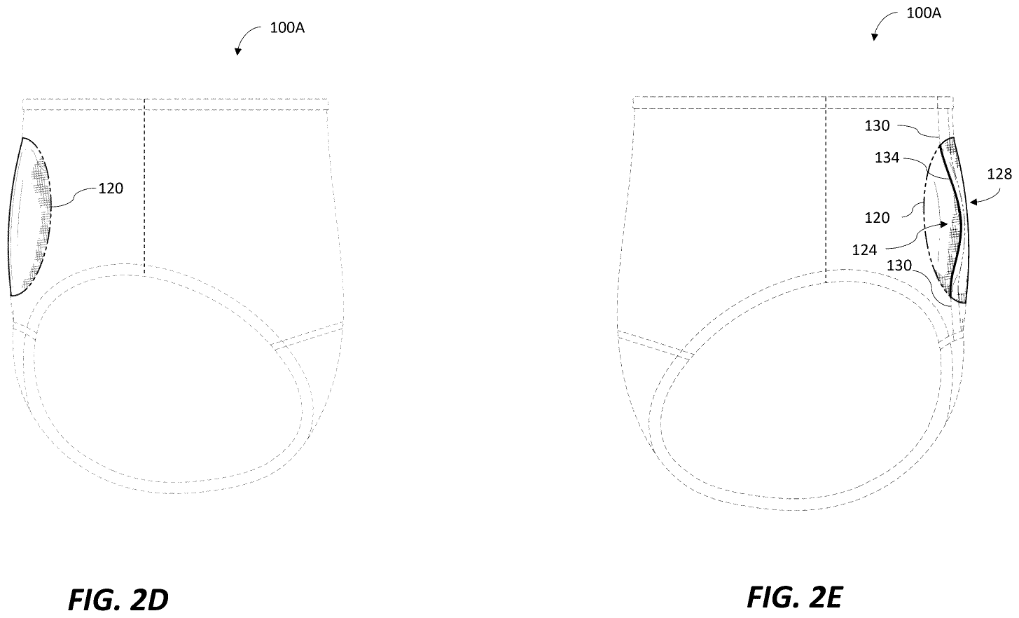

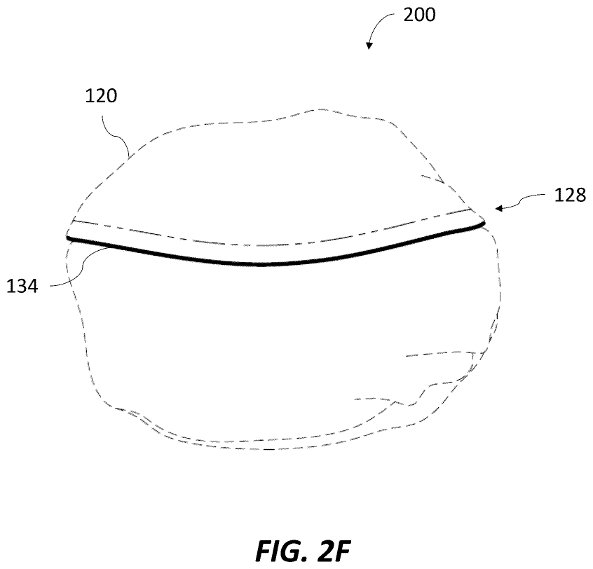

Portions of some of the outer edges of the first layer and the second layer are coupled to or joined to the front panel 104. For example, the portion 132 of the right edge of the first layer 112 is coupled or joined to the front panel 104. Portions of some of the outer edges of the first layer 112 and the second layer 116 are not coupled or joined to the front panel 104. For example, portions 136a of the right side of the second layer 116 are not coupled or joined to the front panel 104. It will also be understood that while portions 136a of the right side of the second layer 116 are illustrated in FIG. 1A, portions of the right side of the second layer 116 are obscured by portions of left side of the first layer 112. An example implementation described below with reference to FIGS. 2A-2E illustrates how portions of the right side of the second layer are obscured from view.

The first layer 112 overlaps with the second layer 116 in implementations of the present disclosure. In the illustrated embodiment, the first layer 112 overlaps with the second layer 116 in the central portion or central region 107 of an exterior side of the front panel 104. In some embodiments, such as that illustrated in FIG. 1A, the first layer 112 can be positioned above (on top of) the second layer 116. In some embodiments (not shown) the second layer 116 can be positioned above (on top of) the first layer 112. In some embodiments, the layers 112, 116 do not overlap in central portion or central region 107 of the front panel 104. The layers 112, 116 can be coupled or joined to the exterior side of the front panel 104. Coupling or joining the layers 112, 116 to the exterior side can help avoid irritation to the skin of the user. In alternative embodiments (not shown), the layers 112, 116 can be coupled or joined to an interior side of the front panel 104 (see, for example, interior side 103 shown in FIG. 2C).

The undergarment 100 includes a seam or weld 120. The seam can join the first layer 112 and the second layer 116 together. In the example illustrated in FIG. 1A, the seam 120 is a circular seam having a center. The left edge 130 of the first layer 112 can be offset from (located a distance away from) the center of the seam 120, rather than, for example, passing through the center of the seam 120. The seam 120 can be positioned in a central portion of the front panel 104 where the first layer 112 overlaps with the second layer 116. The seam 120 can be centered relative to the right and left side of the undergarment 100.

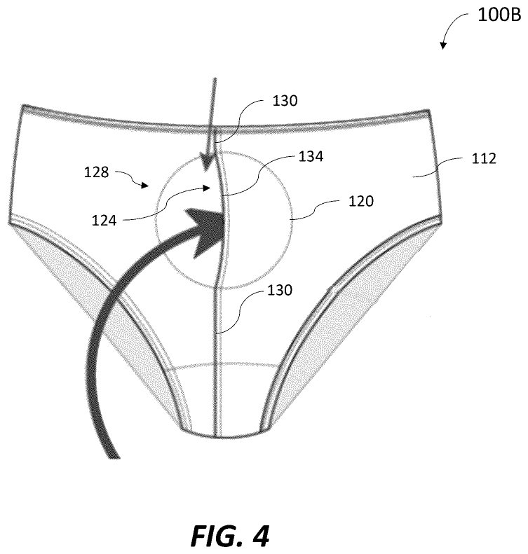

The seam 120 can be any shape. Non-limiting examples include seams that form circular, elliptical, rectangular, square, heart, teardrop, and oval shapes. The shape of the seam 120 can define a shape of the self-contained pouch 200, as shown in FIG. 4, when the undergarment 100 is folded in on itself.

The undergarment 100 can have a pocket guide 128 that can be configured to contain the undergarment 100 when folded in on itself as described herein. In some embodiments, the pocket guide 128 can be defined by the seam 120. The area within the seam 120 can correspond to the overall size of the undergarment 100. For example, the area within the seam 120 can be sized to create a pocket guide 128 that is capable of containing the entire undergarment 100 when the garment is folded in on itself.

The pocket guide 128 can be centered between a right and left edge of the front panel or portion 104. In some embodiments, the pocket guide 128 can be off-centered or not centered between a right and left edge of the front panel or portion 104. The pocket guide 128 can include an opening 124. For example, a portion 134 of the left edge 130 of the first layer can form an opening 124 of the pocket guide 128. other portions of the left edge 130 (for example the portions of the left edge 130 above and below the portion 134) can be secured to the second layer 116 and do not form the opening 124. The other portions of the left edge 130 above and below the portion 134 can be secured to the second layer 116 using any suitable means, for example but not limited to sewing, gluing, and welding the materials. The opening 124 can be off-center from the center of the pocket guide 128. For example, as shown in FIG. 1A, the opening 124 can be positioned closer to the left side of the pocket guide 128. In other embodiments, the opening 124 can be positioned closer to the right side of the pocket guide 128.

In this embodiment, the portion 134 of the left edge 130 of the first layer 112 that forms the opening 124 is generally linear. For example, the portion 134 of the left edge 130 of the first layer 112 is generally straight. In this non-limiting example, the portion 134 of the left edge 130 forming the opening 124 of the pocket guide 128 forms a chord of the circle-shaped pocket guide 128. In some embodiments the portion 134 that forms the opening 124 does not pass through the center of the pocket guide 128, that is, the portion 134 is off-center relative to a center of the pocket guide 128. In some embodiments the portion 134 that forms the opening 124 is centered relative to the pocket guide 128, and can thus form a diameter of the pocket guide 128. It will be understood that pocket guides can take non-circular shapes, and the opening 124 of such pocket guides can pass through, or not pass through, a center of the non-circular pocket guide.

FIGS. 2A-2F illustrate another example embodiment of a garment 100A according to the present disclosure. In this non-limiting example, the garment 100A is an undergarment. Some of the features of the undergarment 100A are similar to features of the undergarment 100 in FIGS. 1A-1B. Therefore, the structure and description of the various features of the undergarment 100 with reference to FIGS. 1A-1B are understood to also apply to the corresponding features of the undergarment 100A in FIGS. 2A-2F, except as described below. Though the features below are described in connection with undergarment 100A, the features can also apply to all other garments according to the present disclosure.

FIGS. 2A-2B illustrate front views of the undergarment 100A. As shown the opening 124 can be positioned off center. For example, the opening 124 can be positioned to the left or the right relative to the center of the pocket guide 128. The opening 124 can be positioned further to the left or right than that which is shown in FIG. 1A. Positioning the opening 124 further to the left or right relative to the center of the pocket guide 128 can provide benefits and advantages including better securing the garment within the pocket guide 128 when folded in on itself as shown in FIG. 2F. This improved securement can also better retain the fluid within the pocket guide 128.

When the opening 124 is positioned further to the left or right, the left edge 130 of the first layer 112 can be positioned further to the left or right (corresponding to the positioning of the opening 124). For example, if the opening 124 is positioned further to the left, the left edge 130 can also be positioned further to the left. In contrast, if the opening 124 is positioned further to the right, the left edge 130 can also be positioned further to the right.

Embodiments of garments according to the present disclosure can include non-linear pocket openings. For example, as shown in FIGS. 2A-2B and 2E, a portion 134 of the leftmost edge 130 of the first layer 112 forming the opening 124 is curved. In some cases, the portion 134 of the leftmost edge 130 of the first layer 112 forming the opening 124 can include a slight curve. In some embodiments, the portion 134 of the leftmost edge 134 of the first layer 112 forming the opening 124 is non-linear. The curved portion 134 of undergarment 100A differs from the portion 134 shown in FIG. 1A, which forms a generally straight line. The portion 134 of the edge 130 can curve from a first location along the seam 120 toward the center of the pocket guide 128 and curve back toward a second location along the seam 120. The first and second locations along the seam 120 can be positioned such that a straight line connecting the two locations would be generally parallel to the longitudinal axis of the user's body when the undergarment 100A is worn, with the apex of the curve extending toward the center of the pocket guide 128. It will be understood that other portions of the left edge 130 of the first layer 112 can be generally straight or linear while the portion 134 is non-linear.

The curve of the portion 134 of the edge 130 can assist in securing the undergarment 100A within the pocket guide 128, when the undergarment 100A is folded in on itself, for example, as shown in FIG. 2F. For example, the curve of the portion 134 of the edge 130 can allow the undergarment 100A to remain in a folded configuration until a user affirmatively unfolds the undergarment 100A. This improved securement can also better retain any fluid within the folded pocket guide 128. Advantageously, the portion 134 of the edge 130 can have a slight curve. The slight curve can remain subtle such that the undergarment 100A remains visually appealing. For example, the subtle nature of the slight curve can reduce or prevent the appearance of a non-smooth finish created by the curve.

While not visible in FIGS. 1A-1B, the interior side 103 of the front panel 104 is visible in FIG. 2C. In some embodiments, the seam 120 does not extend through the front panel 104. For example, the seam 120 may extend through a portion of the front panel 104, but not all layers of the front panel 104. In one non-limiting embodiment, the front panel 104 includes an exterior front panel layer and an interior front panel layer, and the seam 120 passes through the exterior front panel layer but not the interior front panel layer. Explained another way, in some embodiments, the seam 120 does not pass through the interior side of the front panel 104. In examples where the seam 120 does not extend entirely through the front panel 104, the seam 120 is not visible from the interior side of the front panel 104. Implementations of the seam 120 that do not extend entirely through the front panel 104 can help reduce or prevent any irritation to the skin of the user.

FIG. 2F illustrates the undergarment 100A reversibly folded in on itself to form a self-contained pouch 200 wherein the undergarment 100A is contained within the pocket guide 128. As described herein, the shape of the self-contained pouch 200 can be defined by or correspond to the shape of the seam 120. For example, the seam 120 as shown in FIG. 2A is circular. As such, the general shape of the self-contained pouch 200 is circular. The opening 124 being off-center, as shown in FIG. 2A, can assist in keeping the self-contained pouch 200 closed as it provides for a larger overlap in material. Additionally, the off-centered opening 124 can allow for the undergarment 100A to be visually more appealing when worn by the user. The seam 120, as shown in FIG. 2F is the opposite side of the seam 120 that is shown in FIG. 2A. In addition, as described above, the portion 134 of the edge 130 of the first layer 112 is curved, which can assist in maintaining the undergarment 100A in the folded configuration shown in FIG. 2F, thereby assisting in retaining fluids within the folded self-contained pouch 200.

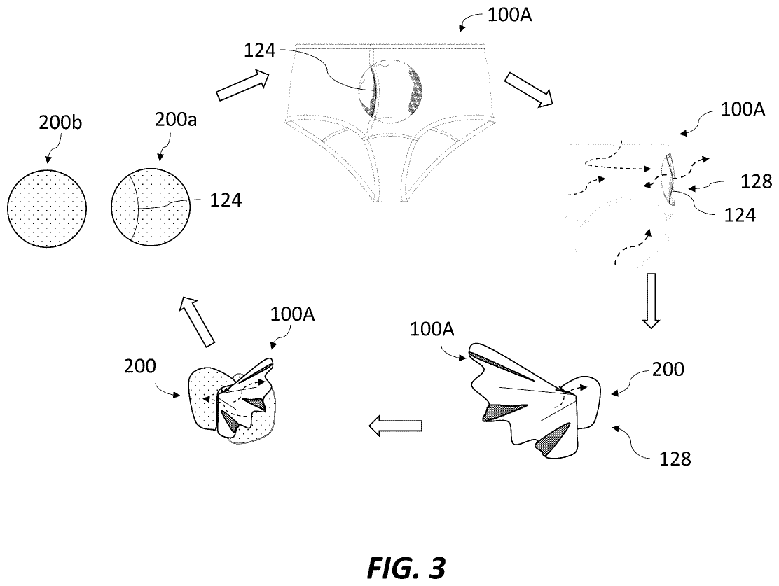

FIG. 3 illustrates a method of folding undergarments according to the present disclosure, for example the undergarment 100A as shown in FIGS. 2A-2F. The opening 124 of the pocket guide 128 can be held opened at the same time a user pushes the back panel 108 of the undergarment 100 inward toward the interior side 103 of the front panel 104. While the user continues to hold the opening 124 open, the user flips the pocket guide inside out while pushing the material of the undergarment into the now inside-out pocket guide 128. To lock the undergarment into the folded configuration as a self-contained pouch, the user can pull the opening 124 over the pouch until it covers more than half of the surface area of the now-inside-out pocket guide 128. The undergarment 100A can be pulled, pushed, or maneuvered into the pocket guide 128.

Once the undergarment 100A is positioned within the pocket guide 128, the self-contained pouch 200 is formed. As described herein, the shape of the self-contained pouch 200 can correspond to the shape of the seam 120. A first side 200a of the self-contained pouch 200 can show the outline of the edge of the opening 124. As shown in FIG. 3 and described above with reference to FIGS. 2A-2E, the portion of the edge of the layer that forms the opening 124 can be curved. A second, opposite side 200b of the self-contained pouch 200 can appear as a solid piece material. Advantageously, the folding of the undergarment 100A is reversible. For example, the undergarment 100A can be folded in on itself and then unfolded. Once the undergarment 100A is folded in on itself, it can then be stored in a way that prevents the soiled components of the used undergarment 100A from transferring to the storage location, for example, a suitcase, purse, bag, etc. To unfold the self-contained pouch, the user opens the opening 124 while pushing the second side 200b towards the first side 200a, causing the pocket guide 128 to reversibly unfold and release the undergarment 100A back out into a substantially flat garment that can be cleaned, stored, and/or worn.

FIG. 4 illustrates another example embodiment of a garment 100B according to the present disclosure. In this non-limiting example, the garment 100B is an undergarment. Some of the features of the undergarment 100B are similar to features of the undergarment 100 in FIGS. 1A-1B and undergarment 100A of FIGS. 2A-2F. Therefore, the structure and description of the various features of the undergarment 100 with reference to FIGS. 1A-1B and undergarment 100A with reference to FIGS. 2A-2F are understood to also apply to the corresponding features of the undergarment 100B in FIG. 4, except as described below. Though the features below are described in connection with undergarment 100B, the features can also apply to all other garments according to the present disclosure.

As illustrated in FIG. 4, the opening 124 of the undergarment 100B can be positioned generally central relative to the pocket guide 128 and/or the undergarment 100B. For example, the opening 124 can be positioned more to the right than that shown in FIGS. 1A, 1B, 2A, and 2B. When the opening 124 is positioned generally central relative to the pocket guide 128 and/or the undergarment 100B, the left edge 130 of the first layer 112 can be positioned generally central relative to the pocket guide 128 and/or undergarment 100B. For example, the opening 124 and the left edge 130 of the first layer 112 can extend along the longitudinal axis of the undergarment 100B.

The garments according to the present disclosure (for example but not limited to undergarment 100, 100A, 100B) can be made of any combination of re-usable and washable materials. The material can be capable of absorbing fluid or fluid-like substances. The material can be leakproof or leak resistant. The material can be configured to absorb menstrual fluid or other bodily fluids such as urine. The interior side of the material can be a quick-drying or moisture-wicking material. The middle portion of the material can be a microfiber or similar material that can be used to trap and contain the bodily fluid during use of the undergarment 100, 100A, 100B. The exterior layer of the material can be impervious and/or waterproof or water resistant to prevent the fluid from traveling through to the exterior of the undergarment and onto or through the exterior apparel (i.e., pants, shorts, skirt, etc.) of the user. The exterior layer of the material may be breathable (for example, allow air to pass through the material), while not allowing fluid to pass through the material. In some embodiments, a gusset can be used to prevent leakage through the undergarment 100, 100A, 100B. The garment can include a compressive material configured to apply a compressive force to the wearer's body. The undergarment 100, 100A, 100B can be used for a variety of purposes. Non-limiting examples include using the garment 100, 100A, 100B as period underwear, a reusable diaper, incontinence underwear, swimwear, swim briefs, briefs, boxer briefs, women's underwear or garments, men's underwear or garments, unisex underwear or garments, and athletic attire (such as but not limited to cycling shorts and yoga attire).

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

While the above detailed description has shown, described, and pointed out novel features of the present disclosure as applied to various embodiments, it will be understood that various omissions, substitutions, and changes in the form and details of the device or process illustrated may be made by those skilled in the art without departing from the spirit of the present disclosure. As will be recognized, the present disclosure may be embodied within a form that does not provide all of the features and benefits set forth herein, as some features may be used or practiced separately from others. All changes which come within the meaning and range of equivalency of the claims are to be embraced within their scope.

The term “comprising” as used herein is synonymous with “including,” “containing,” or “characterized by,” and is inclusive or open-ended and does not exclude additional, unrecited elements or method steps. With respect to the use of substantially any plural and/or singular terms herein, those having skill in the art may translate from the plural to the singular and/or from the singular to the plural as is appropriate to the context and/or application. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

The above description discloses several methods and materials of the present disclosure. The present disclosure is susceptible to modifications in the methods and materials, as well as alterations in the fabrication methods and equipment. Such modifications will become apparent to those skilled in the art from a consideration of this disclosure. Consequently, it is not intended that the present disclosure be limited to the specific embodiments disclosed herein, but that it covers all modifications and alternatives coming within the true scope and spirit of the present disclosure.

1. An undergarment that can be folded in on itself to form a self-contained pouch and un-folded to form a wearable garment, the undergarment comprising:
a front panel comprising an interior side and an exterior side, the exterior side comprising a right portion and a left portion;
a first layer coupled to the front panel along edges of the right portion of the front panel;
a second layer coupled to the front panel along edges of the left portion of the front panel, the first layer overlapping with the second layer in a central portion of the exterior side of the front panel; and
a circular seam joining the first layer, the second layer, and the front panel, the circular seam located in the central portion of the front panel where the first layer overlaps with the second layer, the circular seam defining a shape of the self-contained pouch when the undergarment is folded in on itself.

2. The undergarment of claim 1, wherein a portion of a leftmost edge of the first layer forms an opening within the circular seam.

3. The undergarment of claim 2, wherein the leftmost edge of the first layer that forms the opening comprises a curve.

4. The undergarment of claim 2, wherein the leftmost edge of the first layer is offset a distance from a center of the circular seam, and the opening is offset the distance from the center of the circular seam.

5. The undergarment of claim 1, wherein the circular seam is centered relative to a right side and a left side of the undergarment.

6. The undergarment of claim 1, wherein the circular seam passes through a portion of the front panel.

7. The undergarment of claim 1, wherein the circular seam does not pass through the interior side of the front panel.

8. The undergarment of claim 1, wherein the front panel comprises at least an exterior front panel layer and an interior front panel layer, and wherein the circular seam passes through the exterior front panel layer but not the interior front panel layer.

9. The undergarment of claim 1, wherein the undergarment is a re-usable and washable undergarment configured to absorb menstrual fluids.

10. A garment comprising:
a main body structure comprising a front portion and a back portion;
two layers of fabric coupled to the front portion along outer edges of the front portion; and
a pocket guide configured to form a self-contained pouch when the garment is reversibly folded in on itself, the pocket guide comprising a seam or weld that joins the two layers of fabric and the front portion of the main body structure.

11. The garment of claim 10, wherein a portion of an edge of one of the two layers forms an opening of the pocket guide.

12. The garment of claim 11, wherein the portion of the edge comprises a curve.

13. The garment of claim 10, wherein the two layers of fabric overlap in a central region of the front portion, or wherein the two layers of fabric do not overlap in a central region of the front portion.

14. The garment of claim 10, wherein the two layers of fabric are coupled to an exterior side of the front portion, or wherein the two layers of fabric are coupled to an interior side of the front portion.

15. The garment of claim 10, wherein the pocket guide is circular, elliptical, rectangular, square-shaped, heart-shaped, teardrop-shaped, or oval-shaped.

16. The garment of claim 10, wherein the pocket guide is circular, and wherein the garment comprises an undergarment, a swim garment, or athletic attire comprising a washable material configured to absorb body fluids.

17. The garment of claim 16, wherein an opening of the pocket guide forms a chord that does not pass through a center of the pocket guide, or wherein an opening of the pocket guide forms a diameter of the pocket guide.

18. The garment of claim 10, wherein the pocket guide is centered between a right outer edge and a left outer edge of the front portion, or wherein the pocket guide is not centered between a right outer edge and a left outer edge of the front portion.

19. (canceled)

20. The garment of claim 10, wherein the seam or weld passes through the front portion to an interior-most side of the front portion, or wherein the seam or weld does not pass through the front portion to an interior-most side of the front portion.

21. The garment of claim 10, wherein the main body structure is a single unitary structure, or wherein the main body structure comprises a plurality of portions joined together.

22. (canceled)

23. (canceled)
